(19) United States
(12) Patent Application Publication
(10) Pub. No.: US 2025/0318987 A1
(43) Pub. Date: Oct. 16, 2025

(54) COLD CHAMBER

(71) Applicant: Klafs GmbH, Schwäbisch Hall (DE)

(72) Inventor: Markus Gäbele, Schwäbisch Hall (DE)

(21) Appl. No.: 19/174,564

(22) Filed: Apr. 9, 2025

(30) Foreign Application Priority Data
Apr. 10, 2024 (EP) 24169476.9

(51) Int. Cl. A61H 33/06

(52) U.S. Cl. CPC A61H 33/066; A61H 2033/062

(57) ABSTRACT

A cold chamber is provided with a walk-in interior space that includes a wall cladding. The wall cladding is arranged in front of a wall of the cold chamber in such a way that the wall cladding and the wall form a cavity. The wall is heatable at least partially.

### CROSS REFERENCE

This application claims priority to EP Application Serial No. 24169476.9, filed Apr. 10, 2024, the entirety of which is hereby incorporated by reference.

### FIELD OF THE INVENTION

The present invention relates to a cold chamber.

### BACKGROUND OF THE INVENTION

Cold chambers have been used for many years as part of cryotherapy for medical therapies and to treat athletes. Cold chambers are also becoming increasingly popular in the wellness sector, for example to cool down after a sauna session. Typically, the temperatures generated in the cold chamber are significantly higher than the temperatures of cryotherapy. Slowly cooling down after a sauna session at temperatures between 18° C. and −5° C. is perceived as very pleasant and gentle, especially compared to cooling down with cold water.

Cold chambers in the wellness sector are therefore designed as walk-in rooms, similar to a sauna, in which people can spend a period of time that they find pleasant. The people using the cold chamber can spend the time sitting, lying down or even standing, whereby the ambience in the room is of great importance for a restful and relaxed cool-down. In addition to the prevailing temperature and humidity, the visual design of the cold chamber room, for example in the form of lighting and pictures, has a significant influence on well-being.

A common way to reduce the perceived temperature without increasing the cooling performance is to increases the humidity in the cold chamber, for example by applying a spray mist.

Backlit walls or wall sections are used to create indirect lighting within the cold room and as a special visual element. The backlit wall element forms a kind of cladding element to the actual wall and must be installed at a certain distance from it to allow space for the lighting. This design forms a cavity between the backlit wall and the actual wall.

### BRIEF DESCRIPTION OF THE DRAWINGS

In the following, an example embodiment of the cold chamber is explained in more detail with reference to a drawing.

In the drawing:

FIG. 1 shows a schematic partial side view of an example cold chamber.

### DETAILED DESCRIPTION OF THE DRAWINGS

The design and arrangement of backlit walls can be influenced and limited by the fact that moist air from the cold room gets into the cavity between the backlit wall and the actual wall and leads to unwanted condensation causing the wall to become damp.

It may be desirable to avoid the aforementioned problem and to provide a cold chamber that ensures a flexibility and freedom in design of the backlit wall.

In an example embodiment, a cold chamber is provided with a walk-in interior space comprises a wall cladding, the wall cladding being arranged in front of a wall of the cold chamber in such a way that the wall cladding and the wall form a cavity, the wall being heatable at least partially.

According to an example embodiment, the term “wall” is not necessarily limited to side walls, but also includes ceilings and even floors. The same applies to the respective wall cladding, which accordingly extends to floor claddings und ceiling claddings.

In an example embodiment, the distance between the wall cladding and the wall is 2-30 cm. The dimension applied can vary depending on the specific design of the cold chamber and it represents a compromise between the needed space between the wall cladding and the wall and an interior space as large as possible. The distance may be constant across the height and the width of the wall cladding, meaning that the wall cladding and the wall are parallel to each other. However, the wall cladding may be inclined to the wall so that the distance is not constant but still in the claimed range.

In an example embodiment, the interior space is coolable and the air temperature is lower than the air temperature of the environment of the cold chamber. The air temperature of the interior space may be below 15° C., including but not limited to below 10° C. and/or below 6° C. The set temperature may depend on the respective application and the users' needs. The perceived temperature in the cold chamber depends on both the actual air temperature and the air humidity.

In an example embodiment, the wall cladding is at least partially light-transmissive. The wall cladding may vary in its light transmission rates across its surface, thereby generating patterns. Alternatively, the wall cladding can be uniformly light-transmissive.

According to an example embodiment of the present invention, the wall cladding may comprise acrylic glass. The wall cladding may be formed by an acrylic glass panel.

In an example embodiment, the wall cladding is attached to the ceiling by a suspension device. The wall cladding may be attached to one or two side walls or to install it on the floor of the cold chamber. Alternatively, the wall cladding could also be attached directly to the wall in front of which it is arranged.

The suspension device can separate the cavity from the interior space. However, the cavity may not be sealed so that air can exchange between the interior space and the cavity. Moist air from the interior space can therefore also penetrate into the cavity. If the air is supersaturated, the moisture in the cavity can condense on the wall to cause the to wall become damp. To prevent mold growth, heating elements may therefore be provided to dry the wall and evaporate the moisture if necessary.

In an example embodiment, the cold chamber comprises a light so that the cavity between the wall cladding and the wall can be illuminated. The light may be installed on the ceiling of the cold chamber and may be covered with a cover element to be invisible from the interior space of the cold chamber. In order to illuminate the entire cavity between the wall cladding and the wall, that is, to backlight the wall cladding, the light may be placed behind the wall cladding viewed from the interior space of the cold chamber.

In an example embodiment, the wall cladding may be arranged such that gas exchange occurs between the cavity and the interior space of the cold chamber. Gas exchange is enabled if the cavity between the wall cladding and the wall is not sealed against the interior space. Alternatively, holes may be provided in the wall cladding to create a connection between the cavity and the interior space.

In an example embodiment, the wall cladding may be arranged in front of the wall such that a gap is formed between an upper edge of the wall cladding and a ceiling of the cold chamber and/or between a lower edge of the wall cladding and a floor of the cold chamber, so that gas exchange between the interior space and the cavity occurs via the gap. Accordingly, the wall cladding may not be as wide and/or not as high as the wall so that the cavity is not closed on all sides facilitating gas exchange.

In an example embodiment, the wall may comprise an electric heating element. The wall may be heatable by the electric heating element at least partially in the area forming the cavity. The wall may not necessarily comprise the electric heater in its entirety. It can be either located at a suitable position on the wall or placed outside the cold chamber. It can be sufficient to integrate heating rods into the wall, spaced across a specific area.

Heating the wall causes the wall not to be damped by the high humidity in the cold chamber. Instead, it can be dried regularly or as needed.

In an example embodiment, the wall may comprise pipes through which a heating medium can flow so that the wall is heatable at least partially in an area forming the cavity. The pipes may be configured for a liquid or a gas to flow through them. As a non-limiting example, warm exhaust air from a nearby sauna may be passed through the pipes and thereby heat the wall. This procedure can be effective during the cold chamber closing time.

In an example embodiment, the cold chamber may have a door through which the users of the cold room can enter and leave the cold chamber. The cold chamber may be provided as part of a larger wellness facility that also includes saunas and swimming pools. Due to the regular access of users of the cold chamber who are wet or sweaty, the humidity in the cold chamber may be repeatedly increased, which can lead to the air becoming oversaturated and no longer being able to absorb the moisture. In an example embodiment, the cold chamber may comprise a cooling element on which the humidity can freeze. This cooling element can, for example, be included in the design of the cold chamber and be a visually appealing snow wall. If the cold chamber is heavily frequented, local moisture peaks may occur due to turbulence in the interior space air and that the moisture does not freeze on the snow wall but enters the cavity.

In an example embodiment, the cold chamber may comprise a ventilator that ventilates the cavity between the wall and the wall cladding. Moist air may be extracted at one area of the cavity and dry air may be supplied at another area. The ventilation process may be continuous or discontinuous. In an example embodiment, the cavity may be ventilated only from time to time, for example during the cold chamber closing time.

In an example embodiment, the cold chamber may comprise a drain to drain water from the cavity. For example, the floor in the area of the cavity may slope towards an opening so that the water can drain away there.

In the following FIGURES, identical parts are identified by the same reference signs. Insofar as a FIGURE contains reference signs which are not explicitly referred to in the associated FIGURE description, reference is made to preceding or subsequent FIGURE descriptions.

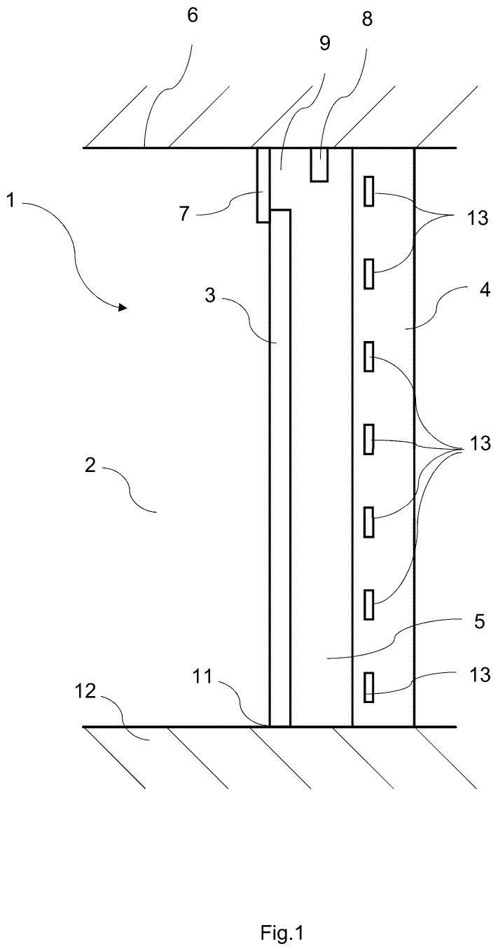

FIG. 1 shows a schematic partial view of a cold chamber 1 having a wall 4 cladding 3 arranged in front of a wall 4, the wall 4 being a side wall of the cold chamber 1. It can also be seen that the wall cladding 3 is not as high as the wall 4 so that a gap 9 forms between an upper edge 10 of the wall cladding 3 and the ceiling 6 and that the cavity 5 formed by the wall cladding 3 and the wall 4 is open at the top. The wall cladding 3 is attached to the ceiling 6 of the cold chamber 1 by means of a suspension device 7. Above the wall cladding 3 the suspension device 7 separates the cavity 5, formed by the wall cladding 3 and the wall 4, from the interior space of the cold chamber 1. Additionally the suspension device 7 covers the light 8 that is located on the ceiling 6 behind the wall cladding 3 viewed from the interior space 2. FIG. 1 also shows heating elements 13 spaced across the height of the wall 4.

The suspension device 7 separates the cavity 5 from the interior space 2. However, the cavity 5 is not sealed so that air can exchange between the interior space 2 and the cavity 5. Moist air from the interior space 2 therefore also penetrates into the cavity 5. If the air is supersaturated, the moisture in the cavity 5 can condense on the wall 4 and the wall 4 becomes damp. To prevent mold growth, heating elements 13 are therefore provided to dry the wall 4 and evaporate the moisture if necessary.

### LIST OF REFERENCE SIGNS

1 cold chamber
2 interior space
3 wall cladding
4 wall
5 cavity
6 ceiling
7 suspension device
8 lighting means
9 gap
10 upper edge of the wall cladding
11 lower edge of the wall cladding
12 floor

1. A cold chamber comprising:
at least one wall;
a walk-in interior space, the walk-in interior space including:
a wall cladding arranged in front of the at least one wall, wherein the wall cladding and the at least one wall form a cavity, the at least one wall being heatable at least partially.

2. The cold chamber according to claim 1, wherein a distance between the wall cladding and the at least one wall is between 2-cm and 30-cm.

3. The cold chamber according to claim 1, wherein the interior space is coolable and the air temperature is lower than the air temperature of the environment of the cold chamber, the air temperature of the interior space being below 15° C.

4. The cold chamber according to claim 1, wherein the wall cladding is at least partially light-transmissive.

5. The cold chamber according to claim 1, wherein the wall cladding comprises acrylic glass.

6. The cold chamber according to claim 1, wherein the wall cladding is attached to a ceiling of the cold chamber by a suspension device.

7. The cold chamber according to claim 1, wherein further comprising a light to illuminate the cavity between the wall cladding and the at least one wall.

8. The cold chamber according to claim 1, wherein the wall cladding allows for gas exchange occurs between the cavity and the interior space of the cold chamber.

9. The cold chamber according to claim 8, wherein the wall cladding is arranged in front of the at least one wall to create a gap between an upper edge of the wall cladding and a ceiling of the cold chamber and/or between a lower edge of the wall cladding and a floor of the cold chamber, so that gas exchange between the interior space and the cavity occurs via the gap.

10. The cold chamber according to claim 1, wherein the at least one wall includes an electric heating element to heat the at least one wall at least partially in the area forming the cavity.

11. The cold chamber according to claim 1, wherein the wall includes pipes through which a heating medium flows so that the wall is heated at least partially in an area forming the cavity.

12. The cold chamber according to claim 1, wherein further comprising a ventilator to ventilate the cavity between the at least one wall and the wall cladding.

13. The cold chamber according to claim 1, wherein further comprising a drain to drain water from the cavity.
